(19) United States
(12) Patent Application Publication
(10) Pub. No.: US 2019/0380667 A1
(43) Pub. Date: Dec. 19, 2019

(54) MEDICAL IMAGE DIAGNOSIS APPARATUS AND CONTROL METHOD

(71) Applicant: Canon Medical Systems Corporation, Otawara-shi (JP)

(72) Inventor: Hiroshi TAKANEZAWA, Nasu (JP)

(73) Assignee: Canon Medical Systems Corporation, Otawara-shi (JP)

(21) Appl. No.: 16/429,112

(22) Filed: Jun. 3, 2019

(30) Foreign Application Priority Data
Jun. 13, 2018 (JP) 2018-112754

(51) Int. Cl. A61B 6/00; A61B 5/055; A61B 6/03; A61B 6/04; G01R 33/48

(52) U.S. Cl. CPC A61B 6/4417; A61B 5/0555; A61B 6/032; G01R 33/481; A61B 6/0407; A61B 6/0457; A61B 6/54; A61B 6/037

(57) ABSTRACT

According to one embodiment, a medical image diagnosis apparatus includes a bed, a first medical image diagnosis device, a second medical image diagnosis device and processing circuitry. The bed supports a table top which is movable in a shorter-side direction of the table top. The first medical image diagnosis device has a first bore and is adjacent to the bed. The second medical image diagnosis device has a second bore and is adjacent to the first medical image diagnosis device, the first bore and the second bore being continuing with each other. The processing circuitry control a position of the table top with respect to the shorter-side direction based on an amount of position gap between the first bore and the second bore.

### CROSS-REFERENCE TO RELATED APPLICATIONS

This application is based upon and claims the benefit of priority from the prior Japanese Patent Application No. 2018-112754, filed Jun. 13, 2018, the entire contents of which are incorporated herein by reference.

### FIELD

Embodiments described herein relate generally to a medical image diagnosis apparatus and a control method.

### BACKGROUND

There is a medical image diagnosis apparatus capable of imaging with multiple modalities, such as an apparatus that combines an X-ray computed tomography (CT) apparatus and a positron emission tomography (PET) apparatus. In the medical image diagnosis apparatus, a gantry of an X-ray CT apparatus and a gantry of a PET apparatus are arranged adjacently to each other in an entry direction of a subject; however, it is difficult to perfectly arrange the mountings so as to align the central axes of the gantries, which thereby causes misalignment.

The misalignment results in position gap between a CT image captured by the X-ray CT apparatus and a PET image captured by the PET apparatus. Therefore, said displacement needs to be corrected by software processing, likely resulting in the degradation of image quality.

### BRIEF DESCRIPTION OF THE DRAWINGS

FIG. 1 is a block diagram illustrating a configuration of a PET-CT apparatus according to the present embodiments.

FIG. 2 is a flowchart illustrating an operation of the PET-CT apparatus according to the present embodiments.

FIG. 3 illustrates an example of a method of computing an amount of misalignment according to the present embodiments.

FIG. 4 is a diagram of the PET-CT apparatus at a time of CT imaging, as viewed from an X-axis direction.

FIG. 5 is a diagram of the PET-CT apparatus at a time of CT imaging, as viewed from a Y-axis direction.

FIG. 6 is a diagram of the PET-CT apparatus at a time of PET imaging, where there is a misalignment in the X-axis direction, as viewed from the X-axis direction.

FIG. 7 is a diagram of the PET-CT apparatus at a time of PET imaging, where there is a misalignment in the X-axis direction, as viewed from the Y-axis direction.

FIG. 8 is a diagram of the PET-CT apparatus at a time of PET imaging, where there is a misalignment in the Y-axis direction, as viewed from the X-axis direction.

FIG. 9 is a diagram of the PET-CT apparatus at a time of PET imaging, where there is a misalignment in the Y-axis direction, as viewed from the Y-axis direction.

FIG. 10 is a diagram of the PET-CT apparatus at a time of PET imaging, where there is a misalignment in the Z-axis direction, as viewed from the X-axis direction.

FIG. 11 is a diagram of the PET-CT apparatus at a time of PET imaging, where there is a misalignment in the Z-axis direction, as viewed from the Y-axis direction.

### DETAILED DESCRIPTION

In general, according to one embodiment, a medical image diagnosis apparatus includes a bed, a first medical image diagnosis device, a second medical image diagnosis device and processing circuitry. The bed supports a table top which is movable in a shorter-side direction of the table top. The first medical image diagnosis device has a first bore and is adjacent to the bed. The second medical image diagnosis device has a second bore and is adjacent to the first medical image diagnosis device, the first bore and the second bore being continuing with each other. The processing circuitry control a position of the table top with respect to the shorter-side direction based on an amount of position gap between the first bore and the second bore.

A medical image diagnosis apparatus and a control method according to the present embodiments will be described below with reference to the drawings. In the embodiments described below, elements assigned with the same reference signs perform the same operations, and redundant descriptions thereof will be omitted as appropriate. Hereinafter, an embodiment will be described with reference to the drawings.

A PET-CT apparatus that combines a PET imaging mechanism and an X-ray CT imaging mechanism will be described below as an example of the medical image diagnosis apparatus. The medical image diagnosis apparatus is not limited thereto; a configuration combining multiple types of imaging apparatuses, a configuration used in the so-called “multimodality imaging”, can also be applied. Examples of such a configuration are a PET-MR apparatus with a PET imaging mechanism and a magnetic resonance (MR) imaging mechanism, a SPECT-CT apparatus with a single photon emission CT imaging mechanism and an X-ray CT imaging mechanism, and an SPECT-MR apparatus with a single photon emission computed tomography (SPECT) imaging mechanism and an MRI imaging mechanism.

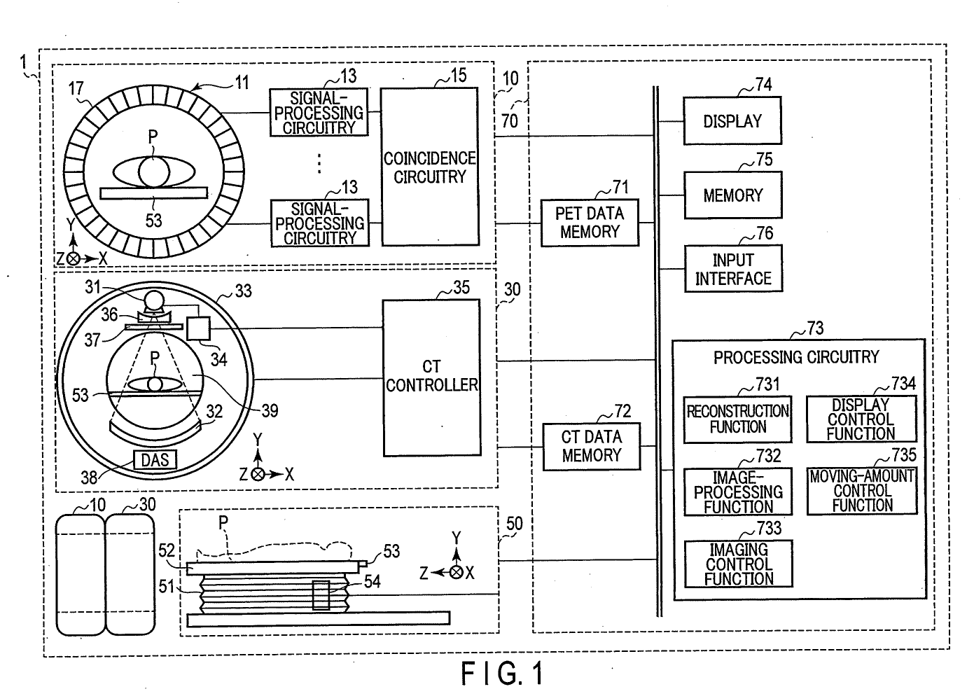

FIG. 1 illustrates a configuration of a PET-CT apparatus 1 according to a first embodiment. As illustrated in FIG. 1, the PET-CT apparatus 1 includes a PET gantry 10, a CT gantry 30, a bed 50, and a console 70. Typically, the PET gantry 10, the CT gantry 30, and the bed 50 are installed in a common examination room, and the console 70 is installed in a control room adjacent to the examination room. The PET gantry 10 is an imaging apparatus that performs PET imaging (PET scan) on a subject P. The CT gantry 30 is an imaging apparatus that performs X-ray CT imaging (CT scan) on the subject P. The bed 50 movably supports a table top 53 on which the subject to be imaged, subject P, is placed. The console 70 is a computer that controls the PET gantry 10, the CT gantry 30, and the bed 50.

For convenience of explanation, FIG. 1 shows a plurality of PET gantries 10 and a plurality of CT gantries 30.

The console 70 is described separately from the PET gantry 10 and the CT gantry 30; however, the console 70 or some of its components may be included in the PET gantry 10 and the CT gantry 30.

As illustrated in FIG. 1, the PET gantry 10 includes a detector ring 11, signal processing circuitry 13, and coincidence circuitry 15.

The detector ring 11 includes a plurality of gamma-ray detectors 17 arranged on a circumference around a central axis Z. The detector ring 11 is accommodated in a housing in which a bore 19, forming an imaging space, is formed. A field of view (FOV) for imaging is set in the bore 19. The subject P is positioned so that an imaged portion of the subject P is included in the FOV. A medicine labeled with positron-emission nuclides is administered to the subject P. Positrons emitted from positron-emission nuclides undergo annihilation with surrounding electrons, and a pair of annihilation gamma rays are generated. The gamma-ray detectors 17 detect annihilation gamma rays emitted from the body of the subject P, and generate an electric signal in accordance with the amount of the detected annihilation gamma rays. For example, the gamma-ray detectors 17 each include a plurality of scintillators and a plurality of photomultipliers. The scintillator receives annihilation gamma rays derived from radioactive isotopes inside of the subject P, and generates light. The photomultiplier generates an electric signal in accordance with the amount of light. The electric signal generated is supplied to the signal processing circuitry 13.

The signal processing circuitry 13 generates single event data based on the electric signals from the gamma-ray detectors 17. Specifically, the signal processing circuitry 13 performs detection time measurement processing, position calculation processing, and energy calculation processing. The signal processing circuitry 13 is implemented by an application-specific integrated circuit (ASIC), a field programmable gate array (FPGA), a complex programmable logic device (CPLD), or a simple programmable logic device (SPLD), configured to execute the detection time measurement processing, position calculation processing, and energy calculation processing.

In the detection time measurement processing, the signal-processing circuitry 13 measures a time at which gamma rays are detected by the gamma-ray detectors 17. Specifically, the signal-processing circuitry 13 monitors a peak value of electric signals from the gamma-ray detectors 17, and measures, as a detection time, a time at which the peak value exceeds a predetermined threshold value. In other words, the signal-processing circuitry 13 electrically detects annihilation gamma rays by detecting that the peak value exceeds the threshold value. In the position calculation processing, the signal-processing circuitry 13 computes an incidence position of the annihilation gamma rays based on the electric signals from the gamma-ray detectors 17. The incidence position of annihilation gamma rays corresponds to positional coordinates of a scintillator that the annihilation gamma rays have entered. In the energy calculation processing, the signal-processing circuitry 13 computes an energy value of the detected annihilation gamma rays based on the electric signals from the gamma-ray detectors 17. Data for the detection time, positional coordinates, and energy value with regard to a single event are associated with one another. A combination of the data for the energy value, positional coordinates, and detection time with regard to a single event is referred to as “single event data”. The single event data is sequentially generated every time annihilation gamma rays are detected. The single event data generated is supplied to the coincidence circuitry 15.

The coincidence circuitry 15 performs coincidence processing on the single event data supplied by the signal-processing circuitry 13. The coincidence circuitry 15 is implemented by an ASIC, a FPGA, a CPLD, or an SPLD, configured to execute the coincidence processing, as a hardware resource. In the coincidence processing, the coincidence circuitry 15 repeatedly specifies single event data related to two single events settled in a predetermined time frame from among single event data which is repeatedly supplied. This pair of single events is estimated to be derived from annihilation gamma rays generated from the same annihilation point. The pair of single events is referred to as a “coincidence event”. A line connecting a pair of gamma-ray detectors 17 (more specifically, scintillators) that have detected the annihilation gamma rays is referred to as a “line of response” (LOR). The event data related to the pair of events constituting the LOR is referred to as “coincidence event data”. The coincidence event data and the single event data are transmitted to the console 70. When the coincidence event data and the single event data need not be distinguished from each other, they are referred to as “PET event data”.

In the above-described configuration, the signal processing circuitry 13 and the coincidence circuitry 15 are included in the PET gantry 10; however, the present embodiments are not limited thereto. For example, the coincidence circuitry 15, or both the signal processing circuitry 13 and the coincidence circuitry 15 may be included in an apparatus separate from the PET gantry 10. A single coincidence circuitry 15 may be provided for all multiple units of signal processing circuitry 13 included in the PET gantry 10, or for each of the grouped multiple units of signal processing circuitry 13 included in the PET gantry 10.

As illustrated in FIG. 1, the CT gantry 30 includes an X-ray tube 31, an X-ray detector 32, a rotation frame 33, a high voltage generator 34, a CT controller 35, a wedge filter 36, a collimator 37, and a data acquisition system (DAS) 38.

The X-ray tube 31 is a vacuum tube that generates X-rays by emitting thermoelectrons from a cathode (filament) to an anode (target) via application of a high voltage and supply of a filament current by the high voltage generator 34. Specifically, X-rays are generated when the thermoelectrons collide with the target. For example, the X-ray tube 31 has a rotating anode-type that generates X-rays by applying thermoelectrons to a rotating anode. The X-rays generated by the X-ray tube 31 are, for example, formed in a cone beam shape via the collimator 37 and applied to the subject P.

The X-ray detector 32 detects X-rays that have been emitted from the X-ray tube 31 and passed through the subject P, and outputs an electric signal corresponding to the amount of X-rays to a DAS 38. The X-ray detector 32 includes, for example, a plurality of X-ray detection element arrays, in which a plurality of X-ray detection elements are arranged in a channel direction along an arc, with a focal point of the X-ray tube 31 as a center. For example, the X-ray detector 32 has an array structure in which a plurality of X-ray detection element arrays (with a plurality of X-ray detection elements arranged in a channel direction) are arranged in a slice direction (row direction).

Specifically, the X-ray detector 32 is, for example, an indirect conversion type detector which includes a grid, a scintillator array, and an optical sensor array.

The scintillator array includes a plurality of scintillators. The scintillator has a scintillator crystal that outputs light having a photon amount corresponding to an amount of incident X-rays.

The grid is arranged on a surface of the scintillator array on an X-ray incident side, and includes an X-ray shielding plate that functions to absorb scattered X-rays. The grid is sometimes called a collimator (one-dimensional collimator or two-dimensional collimator).

The optical sensor array functions to amplify the light received from the scintillator and convert the amplified light into an electric signal, and includes an optical sensor such as a photomultiplier (PMT). The X-ray detector 32 may be a direct conversion type detector with semiconductor elements that convert incident X-rays into electric signals.

The rotation frame 33 supports an X-ray generator and the X-ray detector 32 rotatably about a rotation axis. Specifically, the rotation frame 33 is an annular frame that supports the X-ray tube 31 and the X-ray detector 32, so that the X-ray tube 31 and the X-ray detector 32 face each other, and rotates the X-ray tube 31 and the X-ray detector 32 using the CT controller 35 that will be described later. The rotation frame 33 is rotatably supported by a stationary frame (not illustrated) made of metal such as aluminum. Specifically, the rotation frame 33 is connected to an edge of the stationary frame via a bearing. The rotation frame 33 rotates about the rotation axis Z at a certain angular velocity upon receiving power from a driver of the CT controller 35.

The rotation frame 33 further includes and supports the high voltage generator 34 and the DAS 38 in addition to the X-ray tube 31 and the X-ray detector 32. The rotation frame 33, having such a structure, is accommodated in a housing of a substantially cylindrical shape in which a bore 39 forming an imaging space is formed. The bore substantially matches the FOV. The central axis of the bore matches the rotation axis Z of the rotation frame 33. Detection data generated by the DAS 38 is, for example, transmitted to a receiver (not illustrated) having a photodiode and provided at a non-rotating part (such as a stationary frame; illustration thereof in FIG. 1 omitted) of a mounting apparatus through optical communication from a transmitter having a light-emitting diode (LED), and is transmitted to the console 70. The method of transmitting the detection data from the rotation frame 33 to the non-rotating part of the mounting apparatus is not limited to the aforementioned optical communication. Any method may be adopted in the case of non-contact data transmission.

In the present embodiments, the rotation axis of the rotation frame 33, or the longitudinal direction of the table top 53 of the bed 50 in a non-tilt state, is defined as a “Z-axis direction”; an axial direction which is perpendicular to the Z-axis direction and horizontal to the floor and corresponds to the shorter-side direction of the table top 53 is defined as an “X-axis direction”; and an axial direction which is perpendicular to the Z-axis direction and vertical to the floor is defined as a “Y-axis direction”.

The high voltage generator 34 includes: a high voltage generator including electric circuitry such as a transformer and a rectifier, and generating a high voltage to be applied to the X-ray tube 31 and a filament current to be supplied to the X-ray tube 31; and an X-ray controller that controls an output voltage in accordance with the X-rays emitted by the X-ray tube 31. The high voltage generator may be a transformer type generator, or an inverter type generator. The high voltage generator 34 may be provided to the rotation frame 33 in the CT gantry 30, or to the stationary frame (not illustrated) in the CT gantry 30.

The wedge filter 36 is a filter for adjusting the amount of X-rays emitted from the X-ray tube 31. Specifically, the wedge filter 36 is a filter that allows the X-rays emitted from the X-ray tube 31 to pass therethrough, and attenuates the X-rays so that the X-rays to be applied to the subject P from the X-ray tube 31 exhibits a predetermined distribution. For example, the wedge filter 36 (wedge filter, bow-tie filter) is a filter obtained by processing aluminum so that it has a predetermined target angle and a predetermined thickness.

The collimator 37 is, for example, a lead plate for narrowing the range of radiation of X-rays that have passed through the wedge filter 36, and forms a slit via a combination of a plurality of lead plates and the like. The collimator 37 may be referred to as an “X-ray narrower”.

The DAS 38 reads an electric signal from the X-ray detector 32, and generates digital data (hereinafter also referred to as “raw data”) related to a radiation dose of X-rays detected by the X-ray detector 32 and based on the read electric signal. The raw data is a set of data indicating a channel number and a row number of the X-ray detection elements as a data generation source, a view number indicative of an acquired view (also referred to as “a projection angle”), and a value of the integral of the radiation dose of X-rays detected. The DAS 38 is implemented, for example, via an ASIC on which a circuit element capable of generating raw data is mounted. Said raw data is transmitted to the console 70.

For example, the DAS 38 includes a preamplifier, a variable amplifier, an integration circuit, and an A/D converter for each of the detector pixels. The preamplifier amplifies electric signals from the X-ray detection elements as a connection source at a predetermined gain. The variable amplifier amplifies the electric signals from the preamplifier at a variable gain. The integration circuit integrates the electric signals from the preamplifier for a single-view period to generate an integral signal. The peak value of the integral signal corresponds to a value of the radiation dose of X-rays detected by the X-ray detection elements as a connection source for a single-view period. The A/D converter subjects the integral signal generated by the integration circuit to analog-digital conversion so as to generate raw data.

The CT controller 35 controls the high voltage generator 34 and the DAS 38 to execute an X-ray CT scan in accordance with an imaging control function 733 of processing circuitry 73 of the console 70. The CT controller 35 includes processing circuitry having a CPU, etc., and a driver such as a motor or an actuator. The processing circuitry includes, as hardware resources, a processor such as a CPU or an MPU, and a memory such as a ROM or a RAM. Also, the CT controller 35 may be implemented by an ASIC, FPGA, CPLD, or SPLD.

As illustrated in FIG. 1, the subject P to be scanned is placed on the bed 50 and moved. Said bed 50 is shared by the PET gantry 10 and the CT gantry 30.

The bed 50 includes a base 51, a support frame 52, a table top 53, and a bed actuator 54. The base 51 is a housing that movably supports the support frame 52 in a direction (Y-axis direction) vertical to the floor on which the bed is placed. The base 51 is also movable in the Z-axis direction and the X-axis direction along a rail (not illustrated) mounted on the floor. The support frame 52 is a frame provided above the base 51. The support frame 52 movably supports the table top 53 along the longitudinal direction (Z-axis direction) and the shorter-side direction (X-axis direction). The table top 53 is a plate on which the subject P is placed.

The bed actuator 54 is a motor or an actuator that moves the table top 53 on which the subject P is placed. The bed actuator 54 moves the table top 53 in accordance with the control via the console 70 or the control via the CT controller 35. For example, the bed actuator 54 moves the support frame 52 in the vertical direction (Y-axis direction) so that the body axis of the subject P, placed on the table top 53, matches the central axis of the bore of the rotation frame 33. The bed actuator 54 may also move, in addition to the table top 53, the support frame 52 along the longitudinal direction (Z-axis direction) or the shorter-side direction (X-axis direction) of the table top 53 in accordance with the X-ray CT imaging performed using the CT gantry 30. The bed actuator 54 generates power by being driven at a rotational speed corresponding to the duty ratio, etc., of a driving signal from the CT controller 35. The bed actuator 54 is implemented by a motor such as a direct drive motor or a servo motor.

Namely, the bed 50 supports the table top 53, on which the subject P is placed, so that the table top 53 is movable in three axis directions: the longitudinal direction and the shorter-side direction of the table top 53, and the direction vertical to the floor.

The PET gantry 10 and the CT gantry 30 are arranged adjacently to each other so that the bore of the PET gantry 10 and the bore of the CT gantry 30 are continuing. For example, the PET gantry 10 and the CT gantry 30 are preferably arranged so that the center of the bore of the PET gantry 10 and the center of the bore of the CT gantry 30 substantially match each other. The bed 50 is adjacent to the CT gantry 30, and the long axis of the table top 53 is arranged to be parallel to the central axis Z of the bores of the PET gantry 10 and the CT gantry 30. In the example shown in FIG. 1, the CT gantry 30 and the PET gantry 10 are arranged in the aforementioned order from the side closer to the bed 50; however, said aforementioned order of arranging the CT gantry 30 and the PET gantry 10 may be reversed.

As illustrated in FIG. 1, the console 70 includes a PET data memory 71, a CT data memory 72, processing circuitry 73, a display 74, a memory 75, and an input interface 76. For example, data communication among the PET data memory 71, CT data memory 72, processing circuitry 73, display 74, memory 75, and input interface 76 is performed via a bus.

The PET data memory 71 is a storage device configured to store single event data and coincidence event data transmitted from the PET gantry 10. The PET data memory 71 is a storage device such as a hard disk drive (HDD), a solid state drive (SSD), or an integrated circuit storage device.

The CT data memory 72 is a storage device configured to store CT raw data transmitted from the CT gantry 30. The CT data memory 72 is a storage device such as an HDD, an SSD, or an integrated circuit storage device.

The processing circuitry 73 includes, as hardware resources, a processor such as a CPU, an MPU, or a graphics processing unit (GPU), and a memory such as a ROM or a RAM. By executing various programs read from the memory, the processing circuitry 73 fulfills a reconstruction function 731, an image-processing function 732, an imaging control function 733, a display control function 734, and a moving amount control function 735. The reconstruction function 731, image-processing function 732, imaging control function 733, display control function 734, and moving-amount control function 735 may be implemented by the processing circuitry 73 on a single substrate, or by the processing circuitry 73 on a plurality of substrates to decentralize the functions.

By performing the reconstruction function 731, the processing circuitry 73 reconstructs a PET image representing a distribution of the positron-emitting nuclides applied to the subject P, based on the coincidence event data transmitted from the PET gantry 10. The processing circuitry 73 also reconstructs a CT image representing a space distribution of CT values related to the subject P, based on the CT raw data transmitted from the CT gantry 30. As the image reconstruction algorithm, an existing image reconstruction algorithm such as a filtered back projection (FBP) method or a successive approximation reconstruction method may be adopted. The processing circuitry 73 is capable of generating a positioning image related to PET based on the PET event data, or a positioning image related to CT based on the CT raw data.

By executing the image-processing function 732, the processing circuitry 73 performs various types of image processing on the PET image and the CT image reconstructed by the reconstruction function 731. For example, the processing circuitry 73 performs three-dimensional image processing, such as volume rendering, surface volume rendering, pixel value projection processing, multi-planer reconstruction (MPR) processing, or curved MPR (CPR) processing, on the PET image and the CT image, to generate a display image.

By executing the imaging control function 733, the processing circuitry 73 synchronously controls the PET gantry 10 and the bed 50 to perform a PET scan. The PET scan according to the present embodiments is assumed to be an intermittent movement scan (step-and-shoot technique) in which PET event data is acquired for each acquisition area while the table top 53 is intermittently moved. Also, the processing circuitry 73 synchronously controls the CT gantry 30 and the bed 50 to perform a CT scan. When the PET scan and the CT scan are continuously performed, the PET gantry 10, CT gantry 30, and bed 50 are synchronously controlled. The processing circuitry 73 is also capable of performing a positioning scan by the PET gantry 10 (hereinafter referred to as “PET positioning scan”) and a positioning scan by the CT gantry 30 (hereinafter referred to as “CT positioning scan”). The processing circuitry 73 synchronously controls the PET gantry 10 and the bed 50 to perform the PET positioning scan. The processing circuitry 73 synchronously controls the CT gantry 30 and the bed 50 to perform the CT positioning scan.

By executing the display control function 734, the processing circuitry 73 displays various kinds of information on the display 74. For example, the processing circuitry 73 displays the PET image and the CT image reconstructed via the reconstruction function 731. The processing circuitry 73 also displays a setting window of the acquisition area and the acquisition time.

By executing the moving-amount control function 735, the processing circuitry 73 acquires an amount of position gap indicating the degree of the position gap between the bore of the PET gantry 10 and the bore of the CT gantry 30. The amount of position gap is a value measured in advance when the PET gantry 10 and the CT gantry 30 are installed in a room. The amount of position gap is also a value measured when the PET gantry 10 or the CT gantry 30 are re-installed due to repair of the housing, periodic maintenance, or the like. By executing the moving-amount control function 735, the processing circuitry 73 controls at least the position of the table top 53, with respect to the shorter-side direction of the table top 53, based on the amount of position gap acquired.

Unless otherwise specified, the expression “control the position of the table top 53” includes the following: the table top 53 is moved as the support frame 52 slides the table top 53 in the Z-axis direction and the X-axis direction with respect to the base 51; the table top 53 is moved as the base 51 moves in the Z-axis direction and the X-axis direction without changing the positional relationship between the table top 53 and the base 51; and the support frame 52 and the base 51 collaborate with each other, so that not only does the support frame 52 slide the table top 53, but the base 51 also moves.

In other words, via the moving amount control function 735, the processing circuitry 73 moves the table top 53 by performing the control to move the base 51 (i.e., the control to move the bed 50), the control to move the table top 53, or a combination of the control to move the bed 50 and the control to move the table top 53.

In the case of moving the table top 53, an actuator (not shown) for moving the table top 53, for example, may independently slide the table top 53 in accordance with an instruction from the processing circuitry 73.

If the PET gantry 10 and the CT gantry 30 are configured to be movable, the PET gantry 10 and the CT gantry 30 may be moved to thereby move the table top 53, instead of driving the bed 50 to move the table top 53.

Controlled by the processing circuitry 73 executing the display control function 734, the display 74 displays various kinds of information. For example, a liquid crystal display (LCD), a cathode ray tube (CRT) display, an organic electroluminescence display (OELD), a plasma display, or any other display may be suitably adopted as the display 74. Also, the display 74 may be provided in the housing of the PET gantry 10 or in the housing of the CT gantry 30. The display 74 may be of a desktop type, or configured by a tablet terminal or the like that is capable of wirelessly communicating with the main body of the console 70.

The memory 75 is a storage device such as an HDD, an SSD, or an integrated circuit storage device configured to store various kinds of information. The memory 75 may be a drive configured to read and write various kinds of information from and to, for example, a portable storage medium such as a CD, a DVD, or a flash memory, or a semiconductor memory device such as a random access memory (RAM), other than an HDD or an SSD. The storage area of the memory 75 may be in the console 70 or in an external storage device connected by a network.

The input interface 76 inputs various instructions from the user. Specifically, the input interface 76 is connected to an input device. A keyboard, a mouse, a trackball, a joystick, various switches, a touch pad, a touch panel display, etc., may be used as the input device. The input interface 76 supplies an output signal from the input device to the processing circuitry 73 via a bus. In the present embodiments, the input interface 76 is not limited to be configured to include physical operation parts such as a mouse, a keyboard, a trackball, a switch, a button, a joystick, a touch pad, and a touch panel display. Examples of the input interface 76 also include electric signal processing circuitry that receives an electric signal corresponding to an input operation from an external input device separate from a medical image diagnosis apparatus, and outputs the electric signal to the processing circuitry 73. The input interface 76 may be provided in the CT gantry 30 or in the PET gantry 10. Also, the input interface 76 may be configured by a tablet terminal or the like that is capable of wirelessly communicating with the main body of the console 70.

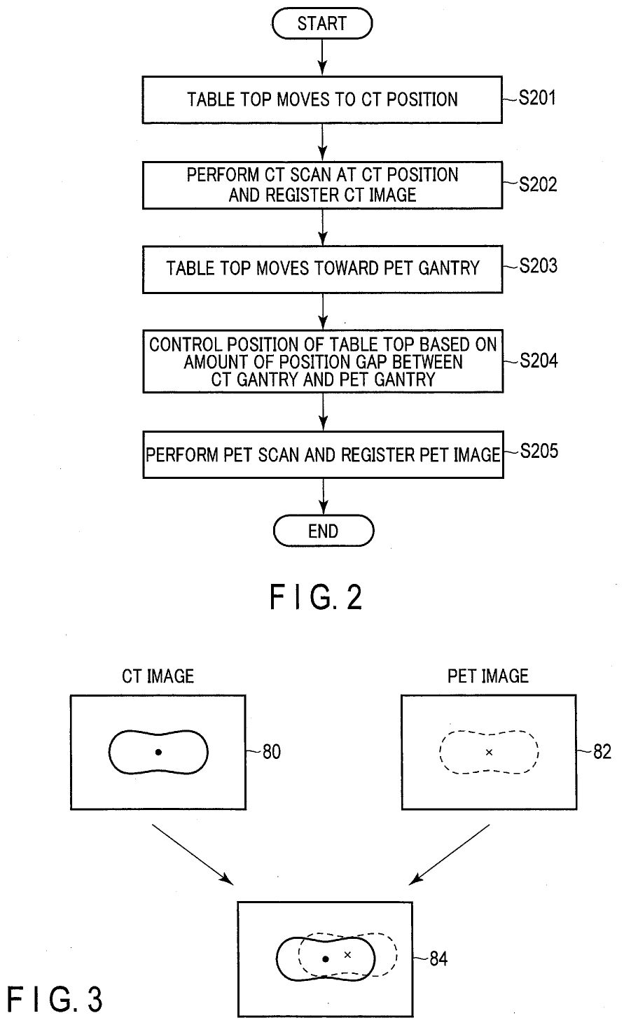

Next, the operation of the PET-CT apparatus according to the present embodiments will be described with reference to the flowchart shown in FIG. 2. In this context, it is assumed that the PET imaging is performed after the CT imaging is performed; however, the CT imaging may be performed after the PET imaging is performed.

In step S201, the bed actuator 54 moves, based on the control by the processing circuitry 73, the table top 53 with the subject P placed thereon to a CT imaging position where the CT imaging is performed on the subject P.

In step S202, the CT controller 35 performs the CT imaging at the position of CT imaging (hereinafter referred to as “CT imaging position”), to generate a CT image. Thereafter, the CT image is registered in the CT data memory 72.

In step S203, upon execution of the control by the bed actuator 54, the table top 53 moves toward the PET gantry 10 so that the PET imaging is performed.

In step S204, by executing the moving-amount control function 735, the processing circuitry 73 controls the bed actuator 54, and controls the position of the table top 53, based on the amount of the position gap between the PET gantry 10 and the CT gantry 30, until the table top 53 moves to a corrected position of PET imaging (hereinafter referred to as “PET imaging position”).

Step S203 and step S204 may be performed as in an indistinguishable sequence of actions.

Namely, the position of the table top 53, based on the amount of position gap, may be controlled at a timing of transition to a PET scan performed using the PET gantry 10, after performance of a CT scan using the CT gantry 30.

In step S205, upon control by the console 70, the PET gantry 10 performs the PET imaging at the PET imaging position, to generate a PET image. Thereafter, the PET image is registered in the PET data memory 71. The operation of the PET-CT apparatus according to the present embodiments ends with the above processing.

An example of the method of computing the amount of position gap will be described with reference to FIG. 3.

It is ideal to arrange the PET gantry 10 and the CT gantry 30 so as to prevent the formation of a position gap between the bore of the PET gantry 10 and the bore of the CT gantry 30, but there may be a position gap of about several millimeters due to an installed condition, temporal change, vibration at the time of imaging, or the like. Even a position gap of a mere several millimeters leads to degradation of image quality.

As an example of computing such an amount of position gap, a phantom is imaged using the CT gantry 30, and the same phantom is imaged using the PET gantry 10, thereby generating a CT image 80 and a PET image 82, as illustrated in FIG. 3.

By executing the image-processing function 732, the processing circuitry 73 generates a composite image 84 formed of the CT image 80 and the PET image 82 superimposed onto each other. The amount of position gap may be computed by comparing the image center of the CT image 80 and the image center of the PET image 82 in the composite image 84.

In the example shown in FIG. 3, it is understood from the composite image 84 that the circle indicating the image center of the CT image 80 and the cross indicating the image center of the PET image 82 do not overlap each other, and that there is a gap between the center of the CT image 80 and the center of the PET image 82. Therefore, the processing circuitry 73, by executing the image processing function 732, may, for example, convert the number of pixels as a difference between the image center of the CT image 80 and the image center of the PET image 82 into Euclidean distance, and calculate the distance as the amount of position gap.

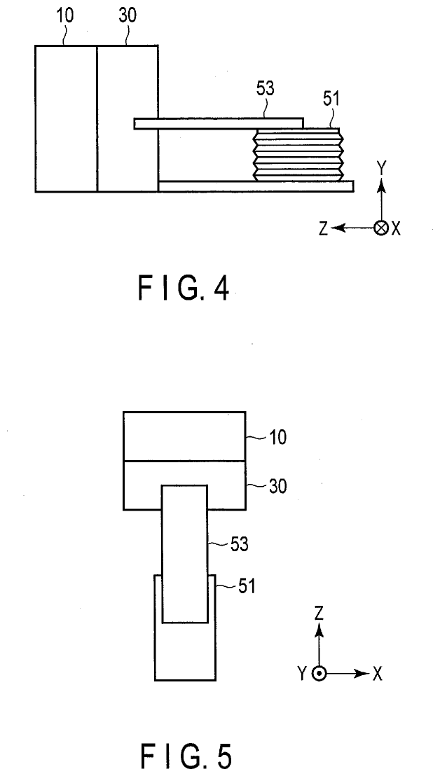

The positional relationship of the table top 53 with the PET gantry 10 and the CT gantry 30 in controlling the moving amount of the bed 50 will be described with reference to FIGS. 4 to 11. In FIGS. 7, 8, 10, and 11, the PET gantry 10 and the CT gantry 30 are arranged with a gap therebetween to the degree that the gap can be visually confirmed, for the convenience of explanation. However, it is also assumed that the position gap cannot actually be visually confirmed.

FIG. 4 is a diagram of the PET-CT apparatus 1 at a time of the CT imaging, as viewed from the X-axis direction. FIG. 5 is a diagram of the PET-CT apparatus 1 at a time of the CT imaging, as viewed from the Y-axis direction.

To make the positional relationship between the bed 50 and each gantry easy to understand, a view of the table top 53 seen from the inside of the bores of the PET gantry 10 and the CT gantry 30 and above the table top 53 on axis Y, is assumed.

When performing the CT imaging, the subject P is placed on the table top 53, and the table top 53 is inserted into the CT gantry 30, to thereby perform the CT scan.

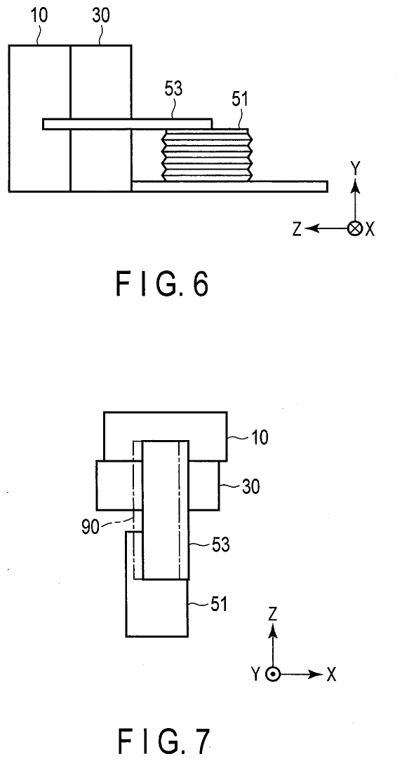

Next, the positional relationship of the table top 53 with the PET gantry 10 and the CT gantry 30, after correcting the amount of position gap and at a time of transition to the PET scan after ending the CT scan, is illustrated in FIGS. 6 and 7. In this context, it is assumed that there is a position gap in the positive X-direction.

FIG. 6 is a diagram of the PET-CT apparatus 1 at a time of the PET imaging, as viewed from the X-axis direction. FIG. 7 is a diagram of the PET-CT apparatus 1 at a time of the PET imaging, as viewed from the Y-axis direction.

In FIGS. 6 and 7, an uncorrected position of inserting the table top (hereinafter referred to as “uncorrected position 90”), when no position gap is assumed, is indicated by a dashed-dotted line. At the uncorrected position 90, the subject is not put into the center of the bore of the PET gantry 10.

Accordingly, the processing circuitry 73, by executing the moving-amount control function 735, controls the bed actuator 54, and controls the position of the table top 53 with respect to the shorter-side direction (X-axis direction) so that it becomes the PET scan position based on the amount of position gap. In this embodiment, the base 51 and the table top 53 move in the positive Z-direction to the position on axis Z being the PET scan position, and based on the amount of position gap, the table top 53 moves in the positive X-direction (the shorter-side direction of the table top 53) where there is a position gap. Specifically, the processing circuitry 73, by executing the moving amount control function 735, generates a control signal including an instruction to move the table top 53 in the positive X-direction based on the amount of position gap. The bed actuator 54 drives an actuator based on the control signal to thereby move the table top 53 in the positive X-direction.

Moving the table top 53 with respect to the position gap in the horizontal X-axis direction, as described above, allows for correction of the position gap between the bore of the PET gantry 10 and the bore of the CT gantry 30.

Instead of moving the table top 53 in two steps by moving the table top 53 in the Z-axis direction and then in the X-axis direction, the table top 53 may be moved in one stage via oblique movement on plane Z-X.

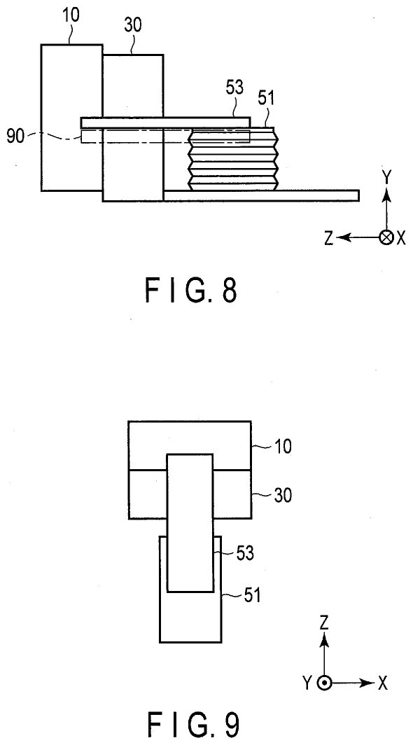

Next, the positional relationship of the table top 53 with the PET gantry 10 and the CT gantry 30, when there is a position gap between the PET gantry 10 and the CT gantry 30 in the positive Y-direction, is illustrated in FIGS. 8 and 9.

FIG. 8 is a diagram of the PET-CT apparatus 1 at a time of the PET imaging, as viewed from the X-axis direction. FIG. 9 is a diagram of the PET-CT apparatus 1 at a time of the PET imaging, as viewed from the Y-axis direction.

Since there is a position gap between the PET gantry 10 and the CT gantry 30 in the positive Y-direction, the subject P is not put into the center of the bore of the PET gantry 10 at the uncorrected position 90.

Therefore, the processing circuitry 73, by executing the moving amount control function 735, controls the bed actuator 54, and controls the position of the table top 53 with respect to the vertical direction so that it becomes the PET scan position based on the amount of position gap. In this embodiment, the base 51 and the table top 53 move in the positive Z-direction to the position on axis Z being the PET scan position, and based on the amount of position gap, the table top 53 moves in the positive Y-direction (the vertical direction) where there is a position gap. Specifically, the processing circuitry 73, by executing the moving amount control function 735, generates a control signal including an instruction to move the table top 53 in the positive Y-direction based on the amount of position gap. The bed actuator 54 drives an actuator based on the control signal and moves the table top 53 in the positive Y-direction.

Instead of moving the table top 53 in two stages by moving the table top 53 in the Z-axis direction and the Y-axis direction, the table top 53 may be moved in one stage by obliquely moving the table top 53 on plane Z-Y.

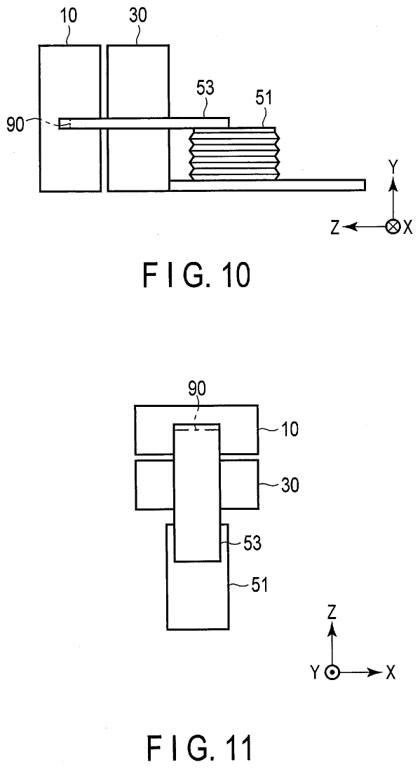

Next, the positional relationship of the table top 53 with the PET gantry 10 and the CT gantry 30, when there is a position gap between the PET gantry 10 and the CT gantry 30 in the positive Z-direction, is illustrated in FIGS. 10 and 11.

FIG. 10 is a diagram of the PET-CT apparatus 1 at a time of the PET imaging, as viewed from the X-axis direction. FIG. 11 is a diagram of the PET-CT apparatus 1 at a time of the PET imaging, as viewed from the Y-axis direction.

Since there is a position gap between the PET gantry 10 and the CT gantry 30 in the positive Z-direction, the subject P is not put into the center of the bore of the PET gantry 10 at the uncorrected position 90 due to the insufficient degree of inserting the table top 53.

Therefore, the processing circuitry 73, by executing the moving amount control function 735, controls the bed actuator 54, and controls the position of the table top 53 with respect to the longitudinal direction of the table top 53 so that it becomes the PET scan position based on the amount of position gap. In this embodiment, the bed 50 and the table top 53 move in the positive Z-direction to the position on axis Z which is the PET scan position.

Specifically, the processing circuitry 73, by executing the moving amount control function 735, generates a control signal including an instruction to the bed actuator 54 to move the table top 53 in the positive Z-direction based on the amount of position gap. The bed actuator 54 drives an actuator based on the control signal and moves the table top 53 in the positive Z-direction.

FIGS. 4 to 11 referred to above describe the position correction according to the amount of position gap in one axis direction. However, the position correction according to the amount of position gap in a plurality of axis directions (such as a case where there is a position gap in two directions, the X-axis direction and the Y-axis direction) may be performed by combining the processing in regard to the respective axis directions.

According to the present embodiments described above, the medical image diagnosis apparatus performs the moving amount control with regard to the position of the table top, based on the amount of the position gap between the center of the bore of the PET gantry and the center of the bore of the CT gantry, so that the amount of the position gap is corrected. In particular, when there is a position gap with respect to the table top in the shorter-side direction of the table top, the table top is moved in the shorter-side direction based on the amount of the position gap.

Thereby, the center of the imaging in the PET scan performed by the PET gantry, and the center of the imaging in the CT scan performed by the CT gantry, can physically match each other. Namely, it is unnecessary to perform image processing on the PET image and the CT image using software and to correct the position gap.

Specifically, according to the medical image diagnosis processing of the present embodiments, the time required for this image processing can be omitted; and since this image processing is not performed, the image quality of the PET image and the CT image can be improved.

The X-ray CT apparatus includes various types such as a Rotate/Rotate type (third-generation CT) in which both the X-ray tube and the detector integrally rotate around the subject P, or a Stationary/Rotate type (fourth-generation CT) in which multiple X-ray detection elements arranged in the form of a ring are stationary and only the X-ray tube rotates around the subject P; and any type can be applied to the present embodiments.

The hardware generating X-rays is not limited to the X-ray tube 31. For example, in place of the X-ray tube 31, the fifth-generation system including a focus coil configured to focus electron beams generated from an electron gun, a deflection coil configured to perform electromagnetic deflection, and a target ring configured to surround a semiperimeter of the subject P and generate X-rays through collision of the polarized electron beams, may be used to generate X-rays.

Furthermore, the present embodiments may be applied to a single-tube type X-ray CT apparatus, and the so-called “multi-tube type X-ray CT apparatus” including a plurality of pairs of X-ray tubes and detectors mounted on a rotating ring.

In the case of the multi-tube type X-ray CT apparatus, the processing circuitry 73 may create the above-described correspondence relationship for each tube, and perform display control processing according to the above-described embodiments based on the longest OLP latency time.

In addition, the functions of the embodiments may be fulfilled by installing a program for executing the processing in a computer such as a work station, and developing the program in a memory. The program that can cause the computer to perform the method may be stored in a storage medium, such as a magnetic disk (e.g., a hard disk), an optical disk (e.g., CD-ROM, DVD), or a semiconductor memory, to be distributed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnosis apparatus comprising:
a bed configured to support a table top which is movable in a shorter-side direction of the table top;
a first medical image diagnosis device having a first bore and that is adjacent to the bed;
a second medical image diagnosis device having a second bore and that is adjacent to the first medical image diagnosis device, the first bore and the second bore being continuing with each other; and
processing circuitry configured to control a position of the table top with respect to the shorter-side direction based on an amount of position gap between the first bore and the second bore.

2. The apparatus according to claim 1, wherein the processing circuitry control the position of the table top with respect to the shorter-side direction, at a timing of transition to a scan performed using one of the first medical image diagnosis device or the second medical image diagnosis device, after performing a scan using remaining one of first medical image diagnosis device or the second medical image diagnosis device.

3. The apparatus according to claim 1, wherein the processing circuitry control a position of the table top with respect to a longitudinal direction.

4. The apparatus according to claim 1, wherein the processing circuitry control a position of the table top with respect to a direction perpendicular to a floor on which the bed is placed.

5. The apparatus according to claim 1, wherein a combination of the first medical image diagnosis device with the second medical image diagnosis device is a combination of an X-ray CT apparatus or an MRI apparatus with a PET apparatus or a SPECT apparatus.

6. The apparatus according to claim 1, wherein the processing circuitry move the table top by performing control to move the bed, control to move the table top, or a combination of the control to move the bed and the control to move the table top.

7. A method of controlling a medical image diagnosis apparatus,
the apparatus comprising:
a bed configured to support a table top which is movable in a shorter-side direction of the table top;
a first medical image diagnosis device having a first bore and that is adjacent to the bed; and
a second medical image diagnosis device having a second bore and that is adjacent to the first medical image diagnosis device, the first bore and the second bore being continuing with each other;
the method comprising controlling a position of the table top with respect to the shorter-side direction, based on an amount of position gap between the first bore and the second bore.

8. The method according to claim 7, comprising controlling the position of the table top with respect to the shorter-side direction, at a timing of transition to a scan performed using one of the first medical image diagnosis device or the second medical image diagnosis device, after performing a scan using remaining one of the first medical image diagnosis device or the second medical image diagnosis device.

9. The method according to claim 7, comprising controlling a position of the table top with respect to a longitudinal direction.

10. The method according to claim 7, comprising controlling a position of the table top with respect to a direction perpendicular to a floor on which the bed is placed.

11. The method according to claim 7, wherein a combination of the first medical image diagnosis device with the second medical image diagnosis device is a combination of an X-ray CT apparatus or an MRI apparatus with a PET apparatus or a SPECT apparatus.

12. The method according to claim 7, wherein the controlling the position of the table top moves the table top by performing control to move the bed, control to move the table top, or a combination of the control to move the bed and the control to move the table top.
